(19) United States
(12) Patent Application Publication
(10) Pub. No.: US 2024/0050644 A1
(43) Pub. Date: Feb. 15, 2024

(54) Active Ingredient Delivery System

(71) Applicant: LTS Lohmann Therapie-Systeme AG, Andernach (DE)

(72) Inventor: Michael Kulik, Urmitz (DE)

(21) Appl. No.: 18/266,740

(22) PCT Filed: Dec. 6, 2021
(86) PCT No.: PCT/EP2021/084424

§ 371 Date: Jun. 12, 2023

(30) Foreign Application Priority Data
Dec. 14, 2020 (DE) 10 2020 133 395.1

(51) Int. Cl. A61M 5/142; A61M 5/172

(52) U.S. Cl. CPC A61M 5/14212; A61M 5/1723

(57) ABSTRACT

An active ingredient means for delivering an active ingredient, in particular a pharmaceutical active ingredient, includes an active ingredient means and an electronic means cooperating therewith. The active ingredient means includes an active ingredient reservoir fluidically connected to a delivery means. The electronic means, which is manufactured in particular as a separate component, includes a control means and a pumping means electrically connected thereto. The pumping means is used to convey the active ingredient from the active ingredient reservoir to the delivery means.

### CROSS-REFERENCE TO RELATED APPLICATIONS

This application is the United States national phase of International Application No. PCT/EP2021/084424 filed Dec. 6, 2021, and claims priority to German Patent Application No. 10 2020 133 395.1 filed Dec. 14, 2020, the disclosures of which are hereby incorporated by reference in their entireties.

### BACKGROUND OF THE INVENTION

### Field of the Invention

The invention relates to an active ingredient delivery system for delivering an active ingredient, in particular a pharmaceutical active ingredient.

### Description of Related Art

For example, transdermal therapeutic systems (TTS) are known for active ingredient delivery. With the aid of transdermal patches, which are adhered to the patient's skin, it is possible to continuously reabsorb a stored active ingredient through the skin. Such systems have the advantage that, unlike injections, active ingredient delivery is painless for the patient.

Furthermore, an active ingredient delivery via microneedles is known. Here, the active ingredient is delivered to the dermis of the skin via a plurality of needles, so that the active ingredient is delivered to the outer layer of the skin. Since there are no nerves in the outer layer of the skin, such active ingredient delivery is also painless for the patient. The microneedles can be designed as hollow needles in which the active ingredient is contained and delivered through the needle openings via the needle tip. Moreover, microneedles are known that consist of a material that dissolves in the skin, so that the active ingredient stored in the needles is delivered to the patient as a result of the dissolution of the needles.

The above systems have been developed for continuous active ingredient delivery, in particular for a longer period of time, but have the disadvantage that, for example, active ingredient delivery at intervals is not possible. For this purpose, such an active ingredient delivery system would have to be removed from the skin and a new such active ingredient delivery system would have to be fixed to the skin for the next active ingredient administration.

An active ingredient delivery system with which active ingredient delivery is also possible at time intervals is known from EP 0 840 634. This active ingredient delivery system comprises a device with microneedles fluidically connected to an active ingredient reservoir. A pump integrated into the system and connected to a control means can be used, for example, to deliver an active ingredient at predetermined times or at predetermined intervals. However, the system described in EP 0 840 634 is a very complex system.

### SUMMARY OF THE INVENTION

It is an object of the invention to provide an active ingredient delivery system for delivering an active ingredient, in particular a pharmaceutical active ingredient, with which an active ingredient can be delivered in a simple and reliable manner, in particular over a longer period of time or at time intervals.

According to the invention, the object is achieved by an active ingredient delivery system having the features as described herein.

The active ingredient delivery system according to the invention is used in particular for delivering a pharmaceutical active ingredient. With the aid of the active ingredient delivery system, it is possible in particular to deliver an active ingredient over a longer period of time. Here, time intervals, points in time, delivery quantities, etc. can be defined.

The active ingredient delivery system comprises an active ingredient means and an electronic means cooperating therewith. The active ingredient means comprises an active ingredient in which the active ingredient, in particular the pharmaceutical active ingredient, is stored. The active ingredient reservoir is fluidically connected to a delivery means. The delivery means is used to deliver the active ingredient to the patient. In particular, the active ingredient is delivered to the dermis of the patient's skin.

The electronic means comprises at least a control means and a pumping means electrically connected thereto. With the aid of the control means, the pumping means can be controlled such that active ingredient is conveyed at predetermined times over a predetermined duration. The pumping means is used to transport the active ingredient from the active ingredient reservoir to the delivery means. Here, the pumping means can act directly or indirectly on the active ingredient reservoir. Moreover, a pump chamber can be arranged between the active ingredient reservoir and the delivery means, on which the pumping means acts.

In a particularly preferred further development of the invention, the active ingredient means and the electronic means are separately formed components. In particular, the active ingredient means is manufactured independently of the electronic means. This has the particular advantage that the active ingredient means can be sterilized in a simple manner without the electronic components of the electronic means being damaged by the sterilization. In this respect, it is particularly possible to sterilize the active ingredient means by irradiation. Furthermore, the separation of the two means has the advantage that the active ingredient means can be manufactured as a foil consisting in particular of several layers. In particular, manufacturing is possible in a continuous process such as a rolling process. This greatly simplifies the manufacture of the active ingredient means.

In particular, compared to the prior art in the form of EP 0 840 634, the separation of the active ingredient means from the electronic means into two separate components is a considerable advantage. In particular, a significantly lower complexity and thus also a more cost-effective manufacture is possible.

Furthermore, the separation of the two means into different components has the advantage that, for example, different electronic means can be connected to the same active ingredient means. In particular, a modular system with different active ingredient means and electronic means can be provided so that different active ingredient delivery systems, for example for different areas of application, can be implemented in a simple manner.

Preferably, the active ingredient means comprises a skin contact surface for applying the active ingredient delivery system to the skin of a patient. In particular, the skin contact surface extends over the entire outer side of the active ingredient means facing the patient and is particularly preferred flat and substantially planar in shape so that good skin contact is ensured. In particular, an adhesive layer may be applied to the skin contact surface so that the active ingredient delivery system can be easily applied to the patient's skin.

Furthermore, it is preferred that the active ingredient means comprises a connecting surface opposite the skin contact surface. The connecting surface is used to connect the active ingredient means to the electronic means. The connecting surface is preferably also planar in shape. If applicable, the connecting surface does not extend over the entire surface, so that, for example, parts of the electronic means can protrude into recesses of the active ingredient means.

It is preferred that both the active ingredient means and the electronic means, except for the electrode components, etc., are made of a plastic material, in particular a plastic foil. The connection between the active ingredient means and the electronic means can thus be made by lamination, for example.

In a preferred further development of the invention, the active ingredient means comprises a receiving area for receiving an active ingredient reservoir, in particular a replaceable active ingredient reservoir. In particular, this has the advantage that the active ingredient means can be manufactured independently of a reservoir containing the active ingredient. Active ingredient reservoirs, for example with different active ingredients, can then be inserted into the receiving area at a later time. This also makes it easy to replace an active ingredient reservoir. This can also be done, for example, while the active ingredient delivery system is arranged on the patient's skin and an empty active ingredient reservoir is replaced with a filled active ingredient reservoir.

Preferably, the receiving area is configured such that it has an opening that is fluidically connected to the delivery means. A check valve can be arranged in said opening so that when an active ingredient reservoir is replaced, no active ingredient can escape from the fluidic connection, i.e. a connecting channel, into the receiving area.

Preferably, a pump chamber is provided between the active ingredient reservoir—regardless of whether the active ingredient reservoir is provided directly in the active ingredient means or a receiving area for an active ingredient reservoir is provided—and the delivery means. The pump chamber is thus fluidically connected to the active ingredient reservoir and the delivery means. The pumping means acts on this pump chamber to convey the active ingredient toward the delivery means. Preferably, a check valve can be provided at the inlet and/or at the outlet of the pump chamber. The check valve provided at the inlet of the pump chamber may be the same check valve arranged in the opening of the receiving area.

In a particularly preferred further development of the invention, the delivery means comprises hollow needles. Preferably, the delivery means comprises a plurality of microneedles formed as hollow needles. In particular, this forms a microneedle array that has at least 4 needles/cm², preferably at least 50 needles/cm², and particularly preferred at least 100 needles/cm². Here, the penetration depth of the needles is preferably such that the needles penetrate into the dermis of the skin, but not deeper into the skin, so as not to penetrate into layers of the skin where there are nerves.

The pumping means is preferably configured and arranged such that it directly acts on the active ingredient reservoir and/or the pump chamber. The pumping effect can be created via a stroke-generating element. The pumping means may comprise a piezoelectric element, an eccentric or the like.

Furthermore, it is preferred that the electronic means comprises a sensor means for acquiring patient data. The sensor means may comprise one or several sensors. The patient data acquired by the sensor means can be transmitted directly to the control unit. It is preferred that the measured patient data is taken into account directly in the control of the pumping means.

For the arrangement of the sensor means, it is preferred that the active ingredient means comprises a recess into which the sensor means protrudes. If necessary, several recesses may also be provided, in each of which one or more sensors are arranged. Providing at least one recess in the active ingredient means has the advantage that the sensors are located close to the skin. If necessary, the active ingredient means may also have an opening in the recess so that the sensor can have direct skin contact.

Further, the electronic means may include a memory means for storing patient data, calculating control signals, and the like. Furthermore, a transmitting and/or receiving means can be provided for data transmission.

It is also preferred that the electronic means comprises an energy storage such as a battery or a capacitor. If applicable, it may be a rechargeable battery. Coupling of electrical and mechanical energy storage systems may also take effect.

### BRIEF DESCRIPTION OF THE DRAWINGS

In the following, the invention is described in more detail by means of a preferred embodiment with reference to the accompanying drawings.

In the drawings:

FIG. 1 is a schematic plan view of an active ingredient delivery system according to the invention,

FIG. 2 is a schematic sectional side view of the active ingredient delivery system according to the invention along line II-II,

FIG. 3 is a schematic view of a pump chamber in different states, and

FIG. 4 is a schematic view of a pumping means.

### DESCRIPTION OF THE INVENTION

The active ingredient delivery system according to the invention comprises an active ingredient means 10 and an electronic means 12. The latter are separately manufactured flat, in particular foil-like components.

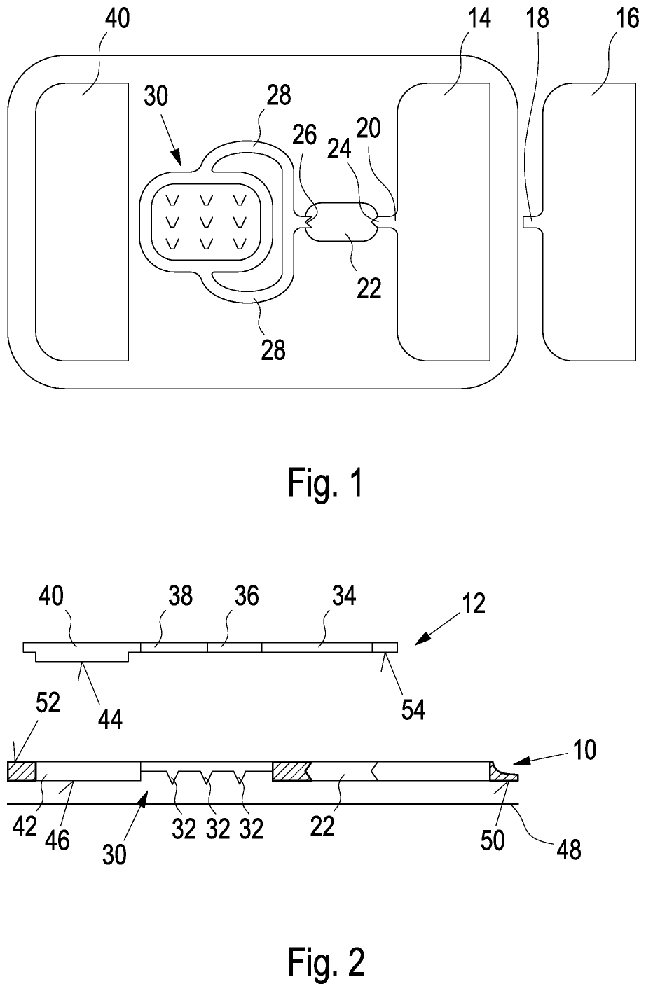

Active ingredient means 10 comprises a receiving area 14 in which a reservoir 16 can be replaceably arranged. In FIG. 1, reservoir 16 can be inserted into receiving area 14 from the right. An outlet 18 of reservoir 16 protrudes into a channel 20 in the inserted state. Channel 20 is initially fluidically connected to a pump chamber 22, with a check valve 24, 26 being arranged at the inlet of pump chamber 22 as well as at the outlet of pump chamber 22. Pump chamber 22 is then fluidically connected via channels 28 to a delivery means 30 for active ingredient delivery.

Delivery means 30 comprises several hollow needles 32 through which the active ingredient in reservoir 16 can be delivered into the patient's skin.

A pumping means 34, which is arranged in the electronic means 12, is provided for conveying the active ingredient from reservoir 16 inserted in receiving area 14 into delivery means 30. When active ingredient means 10 is connected to electronic means 12, pumping means 34 is arranged above pump chamber 22 and acts on pump chamber 22 in such a way that active ingredient is conveyed from reservoir 16 toward delivery means 30. Pumping means 34 is electrically connected to a control means 36. Control means 36, which may also include a memory element, is connected to a battery 38. Furthermore, control means 36 is connected to a sensor means 40. These electronic components are arranged in electronic means 12 (FIG. 2).

Sensor means 40, which may include several sensors, protrudes into a recess 42 provided in agent ingredient means 10. Thus, a bottom 44 of sensor means 40 may be brought into direct contact with the skin, wherein recess 42 may include an opening on its bottom 46 for direct contact with the skin, if necessary.

FIG. 2 shows active ingredient means 10 and electronic means 12 before assembly. Moreover, a cover foil such as a blister 48 is shown. Blister 48 is arranged on a bottom 50 of active ingredient delivery means 10 and is removed immediately before the active ingredient delivery system is placed on the patient's skin. Bottom 50 then forms a skin contact surface, which is provided in particular with an adhesive. Opposite skin contact surface 50, a connecting surface 52 is provided on active ingredient means 10. Connecting surface 52 is used to connect active ingredient means 10 to a bottom 54 of electronic means 12, which thus also serves as a connecting surface. The connection can respectively also be made by adhesion, lamination or the like.

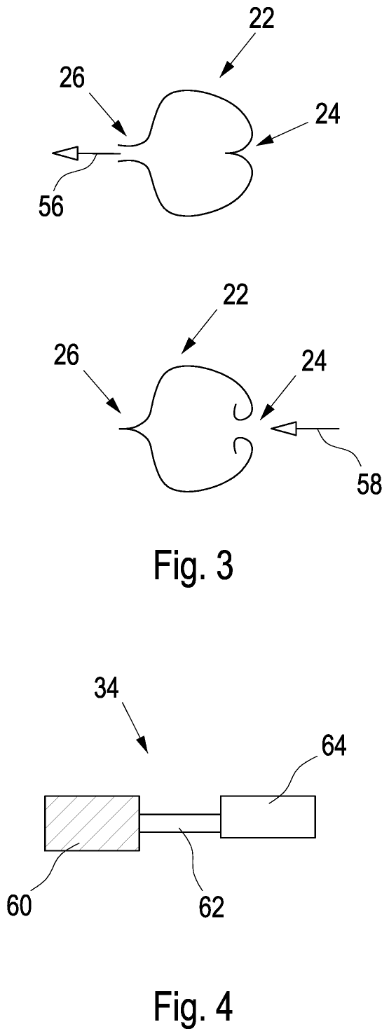

Pump chamber 22 may be formed as shown in FIG. 3 and comprise check valve 24 at the inlet and check valve 26 at the outlet. When the pump presses on chamber 22, valve 24 at the outlet opens so that active ingredient flows out of the chamber in the direction of an arrow 56 toward delivery means 30.

As chamber 22 is filled, valve 26 is closed and valve 24 is opened, allowing fluid to flow from the reservoir in the direction of an arrow 58 into pump chamber 22.

The fluid can be conveyed by a pumping means 34 schematically shown in FIG. 4. It comprises an electric motor 60 connected to an eccentric 64 via an axle 62. Eccentric 64 acts on the top of pump chamber 22 and causes the active ingredient to be conveyed, as described above with reference to FIG. 3.

1. An active ingredient delivery system for delivering an ingredient, in particular a pharmaceutical active ingredient, preferably over a longer period of time and/or time intervals, comprising
an active ingredient means and an electronic means cooperating therewith,
the active ingredient means comprising an active ingredient reservoir fluidically connected to a delivery means, and
the electronic means comprising at least one control means and a pumping means electrically connected thereto, which causes the transport of the active ingredient from the active ingredient reservoir to the delivery means.

2. The active ingredient system according to claim 1, wherein, the active ingredient means and the electronic means are formed as separate components which, in particular, can be manufactured independently of one another.

3. The active ingredient system according to claim 1, wherein the active ingredient means comprises a skin contact surface for applying the active ingredient delivery system to the skin of a patient.

4. The active ingredient system according to claim 3, wherein the active ingredient means comprises a connecting surface opposite the skin contact surface connection to the electronic means.

5. The active ingredient system according to claim 1, wherein the active ingredient means comprises a receiving area for receiving the active ingredient reservoir, in particular a replaceable active ingredient reservoir.

6. The active ingredient system according to claim 1, wherein a pump chamber is provided between the active ingredient reservoir and the delivery means.

7. The active ingredient system according to claim 6, wherein, the pump chamber comprises a check valve at the inlet and/or at the outlet.

8. The active ingredient system according to claim 1, wherein the delivery means comprises hollow needles.

9. The active ingredient system according to claim 1, wherein the pumping means acts on the active ingredient reservoir and/or the pump chamber.

10. The active ingredient system according to claim 1, wherein the electronic means comprises a sensor means for acquiring patient data.

11. The active ingredient system according to claim 10, wherein the patient data are transmitted to the control means and are particularly taken into account when controlling the pumping means.

12. The active ingredient system according to claim 10, wherein the active ingredient means comprises a recess into which the sensor means protrudes.

13. The active ingredient system according to claim 1, wherein the electronic means comprises an energy storage.
